(19) United States
(12) Patent Application Publication
(10) Pub. No.: US 2014/0257102 A1
(43) Pub. Date: Sep. 11, 2014

(54) Devices, Systems, and Methods for Dual Image Intravascular Ultrasound

(71) Applicant: Volcano Corporation, San Diego, CA (US)

(72) Inventor: Norman H. Hossack, Folsom, CA (US)

(73) Assignee: Volcano Corporation, San Diego, CA (US)

(21) Appl. No.: 14/200,496

(22) Filed: Mar. 7, 2014

Related U.S. Application Data
(60) Provisional application No. 61/774,365, filed on Mar. 7, 2013.

(51) Int. Cl. A61B 8/00; A61B 8/12; A61B 8/06

(52) U.S. Cl. CPC A61B 8/445; A61B 8/06; A61B 8/12; A61B 8/4461; A61B 8/463; A61B 8/4416; A61B 8/42
USPC 600/440

(57) ABSTRACT

A system for imaging a vessel of a patient comprises an elongated sheath with proximal and distal ends. The elongated sheath includes a flexible body with a first lumen in communication with a distal opening at the distal end. The system further includes an imaging core disposed within the first lumen and rotatable about an axis of rotation. The imaging core includes a first transducer subassembly adapted to transmit a first beam, distally of the flexible body, through the distal opening. The imaging core also includes a second transducer subassembly adapted to transmit a second beam through the flexible body in a transverse direction to the axis of rotation.

### CROSS REFERENCE TO RELATED APPLICATIONS

The present application claims priority to and the benefit of U.S. Provisional Patent Application No. 61/774,365, filed Mar. 7, 2013, which is hereby incorporated by reference herein in its entirety.

### TECHNICAL FIELD

The present disclosure relates generally to the field of invasive imaging systems, and in particular, to devices, systems, and methods comprising a catheter for dual image intravascular ultrasound (IVUS).

### BACKGROUND

IVUS imaging is widely used in interventional cardiology as a diagnostic tool for assessing a diseased vessel, such as an artery, within the human body to determine the need for treatment, to guide the intervention, and/or to assess its effectiveness. IVUS imaging uses ultrasound echoes to form an image of the vessel of interest. Typically, the ultrasound transducer on an IVUS catheter both emits ultrasound pulses and receives the reflected ultrasound echoes. The ultrasound waves pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound echoes to produce an image of the vessel where the catheter is located.

There are primarily two types of IVUS catheters in common use today: solid-state and rotational. Solid-state IVUS catheters use an array of ultrasound transducers (typically 64) distributed around the circumference of the catheter and connected to an electronic multiplexer circuit. The multiplexer circuit selects array elements for transmitting an ultrasound pulse and receiving the echo signal. By stepping through a sequence of transmit-receive pairs, the solid-state IVUS system can synthesize the effect of a mechanically scanned transducer element, but without moving parts. Since there is no rotating mechanical element, the transducer array can be placed in direct contact with the blood and vessel tissue with minimal risk of vessel trauma and the solid-state scanner can be wired directly to the imaging system with a simple electrical cable and a standard detachable electrical connector.

In the typical rotational IVUS catheter, a single ultrasound transducer element fabricated from a piezoelectric ceramic material is located at the tip of a flexible driveshaft that spins inside a sheath inserted into the vessel of interest. The typical transducer element is oriented such that the ultrasound beam propagates generally perpendicular to the axis of the catheter. In the typical IVUS catheter, the fluid-filled (e.g., saline-filled) sheath protects the vessel tissue from the spinning transducer and driveshaft while permitting ultrasound signals to freely propagate from the transducer into the tissue and back. As the driveshaft rotates (typically at 30 revolutions per second), the transducer is periodically excited with a high voltage pulse to emit a short burst of ultrasound. The ultrasound is emitted from the transducer, through the saline-fill and sheath wall, in a direction generally perpendicular to the axis of rotation of the driveshaft. The same transducer then listens for the returning echoes reflected from various tissue structures, and the IVUS imaging system assembles a two dimensional image of the vessel cross-section from a sequence of several hundred of these ultrasound pulse/echo acquisition sequences occurring during a single revolution of the transducer.

Current methods for treating vascular occlusions involve crossing the occlusion with a guide wire prior to opening the occluded vasculature. A true lumen (i.e., natural vascular lumen) crossing technique involves passing the guidewire through the occlusion. Using current IVUS systems for guide wire placement, clinicians are often unable to navigate the IVUS catheter through the occlusion and remain within the true lumen. Sub-intimal crossing is another technique for guide wire placement that involves advancing the guidewire through a sub-intimal passage, tangentially past the occlusion. The guide wire re-enters the true lumen after passing the occlusion. Both of these techniques carry the risk of vasculature perforation because the clinician is unable to visualize the true lumen on the opposite side of the occlusion and, as a consequence, may direct the IVUS catheter toward or through the vascular wall.

While existing IVUS catheters deliver useful therapeutic information, there is a need for improved imaging capability to assist navigation, vasculature evaluation, and delivery of treatment.

### SUMMARY

The embodiments of the invention are summarized by the claims that follow below.

In one embodiment, system for imaging a vessel of a patient comprises an elongated sheath with proximal and distal ends. The elongated sheath includes a flexible body with a first lumen in communication with a distal opening at the distal end. The system further includes an imaging core disposed within the first lumen and rotatable about an axis of rotation. The imaging core includes a first transducer subassembly adapted to transmit a first beam, distally of the flexible body, through the distal opening. The imaging core also includes a second transducer subassembly adapted to transmit a second beam through the flexible body in a transverse direction to the axis of rotation.

In another embodiment, a method of imaging a vessel of a patient comprises providing an elongated sheath having a proximal end and a distal end. The sheath includes a flexible body with a first lumen in communication with a distal opening at the distal end. The method further includes rotating a first transducer subassembly about an axis of rotation within the first lumen while directing a first beam from the distal opening and rotating a second transducer subassembly about the axis of rotation within the first lumen while directing a second beam approximately transversely to the axis of rotation. The method further includes receiving reflected first and second beams.

Additional aspects, features, and advantages of the present disclosure will become apparent from the following detailed description.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an IVUS system according to one embodiment of the present disclosure.

FIG. 2 is an illustration of a distal end of a multi-scan imaging system with a forward-scanning transducer subassembly and a side-scanning transducer subassembly, according to an embodiment of the present disclosure.

FIG. 3 is an illustration of the distal end of a multi-lumen catheter according to one embodiment of the present disclosure.

FIG. 4 is a side view of the imaging areas of the multi-scan imaging system of FIG. 2.

FIG. 5 is a perspective view of the imaging areas of the multi-scan imaging system of FIG. 2.

FIG. 6 is a side view of a distal end of a multi-lumen catheter and imaging core illustrating an imaging area scanned by the forward-scanning transducer subassembly and an imaging area scanned by the side-scanning transducer subassembly, according to an embodiment of the disclosure.

FIG. 7 is a flowchart describing a method of using an IVUS catheter system.

FIG. 8 is an illustration of a multi-scan imaging system extended within and generally parallel to a lumen of a patient anatomy.

FIG. 9 is an illustration of the multi-scan imaging system of FIG. 8 extended within and generally oblique to the lumen of the patient anatomy.

FIG. 10 is an illustration of synchronized forward-scanning and side-scanning transducer images.

FIG. 11 is an illustration of desynchronized forward-scanning and side-scanning transducer images.

FIG. 12 is an illustration of an IVUS catheter system according to one embodiment of the present disclosure.

FIG. 13 is an illustration of a distal end of the IVUS catheter system of FIG. 10.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

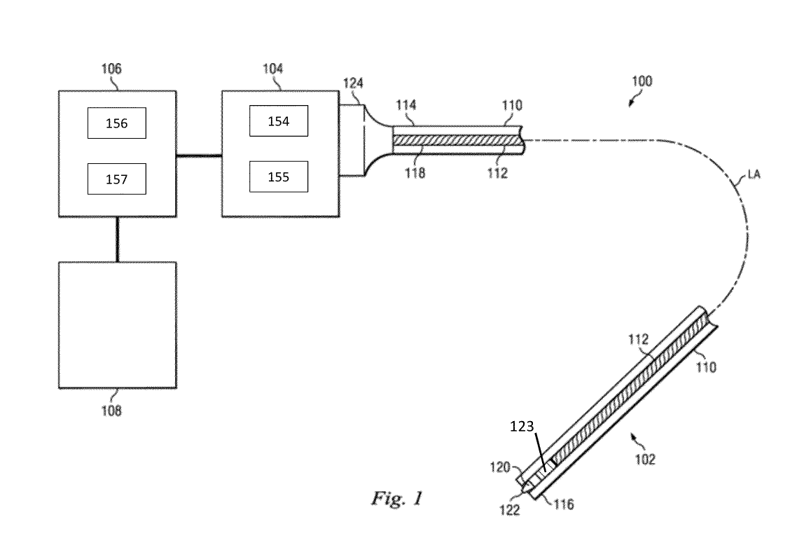

Referring first to FIG. 1, an imaging system 100 for insertion into a patient for diagnostic imaging is shown. The system 100 comprises an IVUS catheter 102 coupled by a patient interface module (PIM) 104 to an IVUS control system 106. The control system 106 is coupled to a monitor 108 for display of an IVUS image.

The IVUS catheter 102 includes an elongated, flexible catheter sheath 110 shaped and configured for insertion into a lumen of a blood vessel (not shown) such that a longitudinal axis LA of the catheter 102 substantially aligns with a longitudinal axis of the vessel at any given position within the vessel lumen. In that regard, the curved configuration illustrated in FIG. 1 is for exemplary purposes and in no way limits the manner in which the catheter 102 may curve in other embodiments. Generally, the catheter 102 may be configured to take on any desired arcuate profile when in the curved configuration.

In some embodiments, a rotating imaging core 112 extends within the sheath 110. Accordingly, in some embodiments imaging core 112 is rotated relative to the sheath 110. The sheath 110 has both a proximal end portion 114 and a distal end portion 116. The imaging core 112 has a proximal end portion 118 disposed within the proximal end portion 114 of the sheath 110 and a distal end portion 120 disposed within the distal end portion 116 of the sheath 110.

The distal end portion 116 of the sheath 110 and the distal end portion 120 of the imaging core 112 are inserted into a patient during the operation of the system 100. The usable length of the catheter 102 (e.g., the portion that can be inserted into a patient) can be any suitable length and can be varied depending upon the application.

The proximal end portion 114 of the sheath 110 and the proximal end portion 118 of the imaging core 112 are connected to the interface module 104. The proximal end portions 114, 118 are fitted with a catheter hub 124 that is removably connected to the interface module 104.

The distal end portion 120 of the imaging core 112 includes a transducer subassembly 122 and a transducer subassembly 123. The transducer subassemblies 122, 123 can be of any suitable type for visualizing a vessel and, in particular, a severe occlusion in a vessel. In this embodiment, the transducer subassembly 122 is configured for forward scanning to capture images of the vessel area distal of the transducer subassembly 122. In this embodiment, the transducer subassembly 123 is configured for side scanning to capture images of a cross-sectional area of the vessel. The transducer subassemblies 122, 123 are configured to be rotated (either by use of a motor or other rotary device) to obtain the images of the vessel. Accordingly, the transducer subassemblies may be an ultrasound transducer array (e.g., arrays having 16, 32, 64, or 128 elements are utilized in some embodiments) or single ultrasound transducers. In alternative embodiments, one or more optical coherence tomography (“OCT”) elements (e.g., mirror, reflector, and/or optical fiber) may be included in or comprise the transducer subassembly. Suitable transducer subassemblies may include, but are not limited to, one or more advanced transducer technologies such as Piezoelectric Micromachined Ultrasonic Transducer (“PMUT”) and Capacitive Micromachined Ultrasonic Transducer (“CMUT”).

The rotation of imaging core 112 within sheath 110 is controlled by PIM 104. For example, PIM 104 provides user interface controls that can be manipulated by a user. In some embodiments PIM 104 may receive and analyze information received through imaging core 112. It will be appreciated that any suitable functionality, controls, information processing and analysis, and display can be incorporated into PIM 104. Thus, PIM 104 may include a processor circuit 154 and a memory circuit 155 to execute operations on catheter 102 and receive, process, and store data from catheter 102. In some embodiments, PIM 104 receives data from ultrasound signals (echoes) detected by imaging core 112 and forwards the received echo data to control system 106. Control system 106 may include a processor circuit 156 and a memory circuit 157 to execute operations on catheter 102 and receive, process, and store data from catheter 102. In some embodiments, PIM 104 performs preliminary processing of the echo data prior to transmitting the echo data to control system 106. PIM 104 may perform amplification, filtering, and/or aggregating of the echo data, using processor circuit 154 and memory circuit 155. PIM 104 can also supply high- and low-voltage DC power to support operation of catheter 102 including the circuitry within transducer assembly 122.

In some embodiments, wires associated with IVUS imaging system 100 extend from control system 106 to PIM 104. Thus, signals from control system 106 can be communicated to PIM 104 and/or vice versa. In some embodiments, control system 106 communicates wirelessly with PIM 104. Further according to some embodiments, catheter 102 may communicate wirelessly with PIM 104. Similarly, it is understood that, in some embodiments, wires associated with the IVUS imaging system 100 extend from control system 106 to monitor 108 such that signals from control system 106 can be communicated to monitor 108 and/or vice versa. In some embodiments, control system 106 communicates wirelessly with monitor 108.

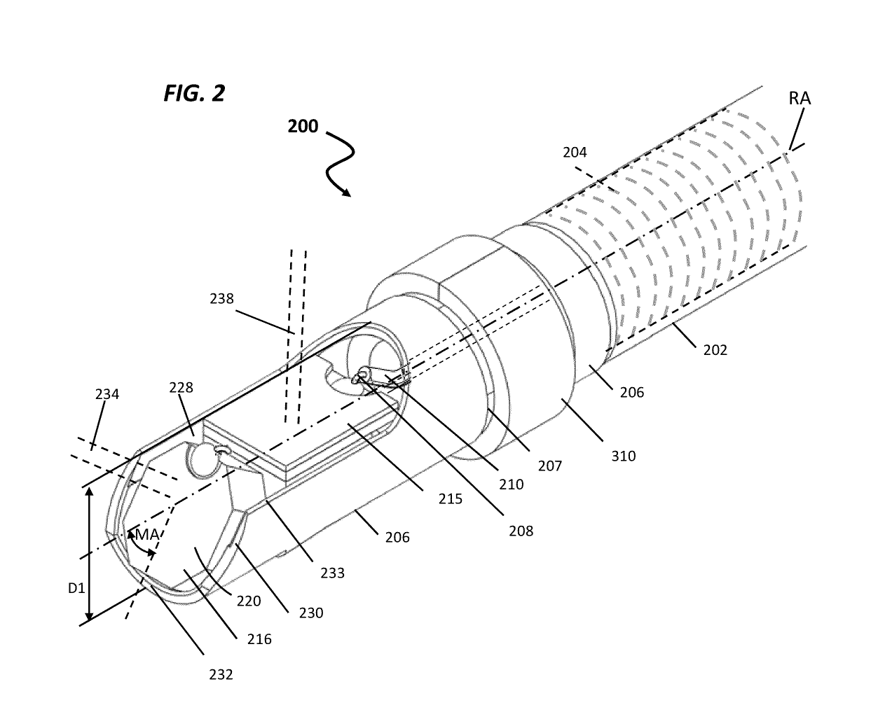

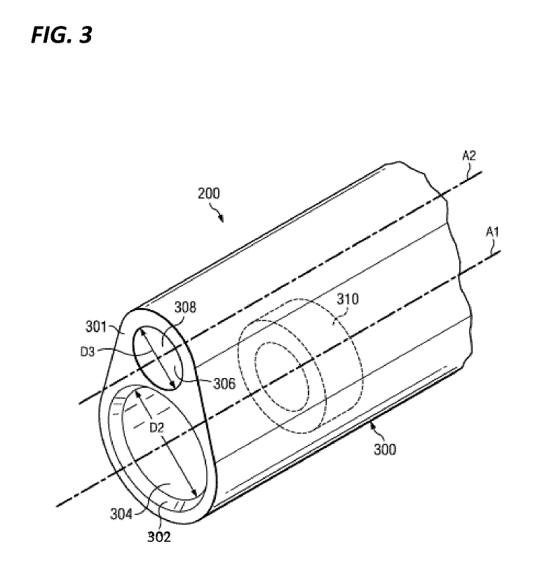

FIG. 2 provides a detailed view of a distal portion of a rotational IVUS catheter system 200 which may be similar to IVUS catheter 102. As shown in FIGS. 2 and 3, the catheter system 200 includes an imaging core 202. The imaging core 202 includes a flexible drive shaft 204 which may be formed from two or more layers of counter wound stainless steel wires, welded or otherwise secured to a transducer housing 206 such that rotation of the flexible drive shaft 204 imparts rotation to the housing 206. A transducer housing 206 houses a side-scanning transducer subassembly 215. In some aspects, this transducer subassembly 215 may be similar to the side-scanning transducers of the Revolution® catheter system available from Volcano Corporation and described in U.S. Pat. No. 8,104,479, or those disclosed in U.S. Pat. Nos. 5,243,988 and 5,546,948, each of which is hereby incorporated by reference in its entirety. The transducer housing 206 also houses a forward-scanning transducer subassembly 216. The transducer housing 206 includes a waist section 207. In some aspects, this transducer subassembly may be similar to the forward-scanning transducer described in U.S. Provisional App. No. 61/658,748 which is hereby incorporated by reference in its entirety. As described further below, transducer subassemblies 215, 216 are coupled to the flexible drive shaft 204 such that rotational motion of the drive shaft imparts rotational motion to the transducer subassemblies.

Electrical cables 208 with optional shielding 210 extends through an inner lumen of the flexible drive shaft 204. Leads from one of the cables 208 are soldered, welded, or otherwise electrically coupled to a transducer subassembly 215. At least a portion of the cables 208 further extends past the subassembly 215, and the leads of another one of the cables 208 are soldered, welded, or otherwise electrically coupled to a transducer subassembly 216. The proximal end of the cables 208 terminate in a series of rings for electrical interface with an interface module through a hub (similar to hub 124).

The transducer subassemblies 215, 216 are secured to the housing 206 by a backing material such an epoxy or a similar bonding agent. The backing material may also serve to absorb acoustic reverberations within the housing 206 and as a strain relief for the electrical cable 208 where it is attached to the transducer subassemblies.

The transducer subassembly 216 includes a generally circular or elliptical planar face 220 mounted at an oblique mounting angle MA with respect to a rotational axis RA of the imaging core 202. In one embodiment, the mounting angle MA may be 45°, but larger or smaller mounting angles may also be suitable. For example, mounting angles of 55°, 35°, or 15° with respect to the rotational axis may be suitable depending upon the desired field of view.

The transducer housing 206 includes an opening 228 bounded by a wall section 230, a wall section 232, and a wall section 233. The wall section 230 is angled with respect to the rotational axis RA and may, for example, be angled at the mounting angle MA. The wall section 232 may be generally transverse to the rotational axis RA. The wall section 233 may be generally parallel to the rotational axis RA and/or parallel to the planar face of the transducer assembly 215. The transducer housing 206 has an outer diameter D1.

In use, as configured, the transducer subassembly 216 produces a forward-imaging ultrasound beam 234 propagating generally perpendicular to the face 220 of the transducer subassembly 216. The beam 234 passes through the opening 228 of the transducer housing 206 and propagates distally from the catheter system 200 into a vascular region distal (i.e., forward) of the catheter. After reflecting off tissue, including blockages or occlusions, located distally of the transducer subassembly 216, an echo beam is detected by the transducer subassembly and sent to a control system for processing and display.

The side-scanning transducer subassembly 215 may include, for example, a PMUT MEMS transducer layer arranged approximately parallel to the rotational axis RA, with a spherically focused portion facing the opening 228. In some embodiments, transducer subassembly 215 may include an application-specific integrated circuit (ASIC) (not shown) within the imaging core 202. The ASIC may be electrically coupled to the transducer layer. In some embodiments of the present disclosure the ASIC may include an amplifier, a transmitter, and a protection circuit associated with the transducer layer. In some embodiments, the ASIC is flip-chip mounted to a substrate of the PMUT MEMS transducer layer using anisotropic conductive adhesive or suitable alternative chip-to-chip bonding method.

In use, as configured, the transducer subassembly 215 produces a side-imaging ultrasound beam 238 propagating generally perpendicular to the face of the transducer subassembly 215. The beam 238 passes through the opening 228 and propagates radially away from the catheter system 200 into a lateral vascular region. After reflecting off tissue, including blockages or occlusions, located laterally of the transducer subassembly 215, an echo beam is detected by the transducer subassembly and sent to a control system for processing and display.

Referring now to FIG. 3, the catheter 200 also includes an elongated sheath 300 formed of a flexible, biologically compatible material which may include metals, plastics, and/or ceramics. Because the ultrasonic beam 238 propagates through the sheath 300, the sheath may be formed of a material with an acoustic impedance and sound speed particularly well-suited for conducting the ultrasound beam from the transducer out into the blood vessel with minimal reflection, attenuation, or beam distortion.

The sheath 300 includes a distal wall 301 having a distal opening 302 in communication with a lumen 304 that extends the length of the sheath 300. The lumen 304 is centered about a longitudinal axis A1 and has a diameter D2 which is sufficiently larger than the outer diameter D1 of the transducer housing 206 to permit rotation of the transducer housing and transducer subassemblies 215, 216 within the lumen 304. In one embodiment, for example, the diameter D2 of the lumen 302 may be approximately 0.035-0.020 inches. The distal wall 301 extends generally perpendicular to the longitudinal axis A1.

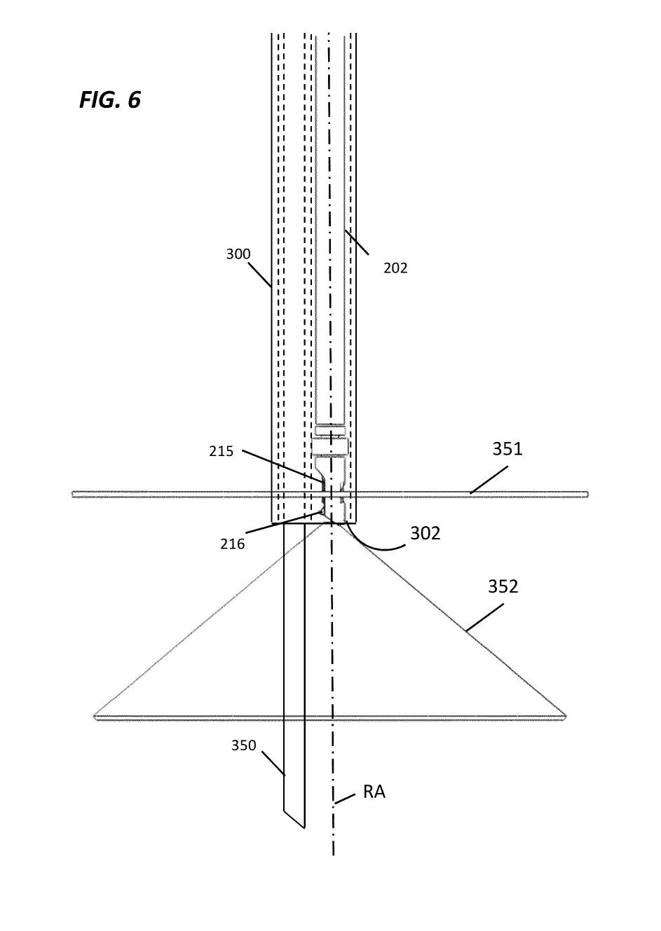

The imaging core 202, including transducer housing 206 and transducer subassemblies 215, 216 are inserted into and rotate within the lumen 304. (FIG. 6) As assembled, the rotational axis RA of the transducer subassemblies 215, 216 is generally coincident with the longitudinal axis A1 of the lumen 302. Also as assembled, the wall section 232 of the housing 206b is generally flush with the distal wall 301 to prevent the rotating housing or transducer subassembly 216 from contacting tissue, blood, or other bodily fluids surrounding the sheath 300. In other embodiments, the housing 206 may be slightly extended out of or retracted into the lumen 304. To prevent longitudinal migration of the transducer subassemblies 215, 216 within the lumen 304, the housing 206 is rotationally coupled to the sheath 300. In this embodiment, the ring shaped insert 310, is attached around the waist section 207 of the housing 206, such that the waist section rotates with respect to the insert 310. The insert 310 may include a low melt material that is fused to the wall of the lumen 304 and is thus stationary with respect to the lumen 304. In this way, the housing 206 may be rotated within the lumen 304 relative to the insert 310. At the same time, the insert 310 engages the housing 206 to prevent forward or rearward migration of the housing, and therefore the transducer subassemblies 215, 216, within the lumen 304.

The sheath 300 also includes an opening 306 in the distal wall 301. The opening 306 is in communication with a lumen 308 that may extend the length of or a partial length of the sheath 300. The lumen 308 may be sized to receive a guide wire 350 (See FIG. 6) or a wire for conducting a therapeutic procedure. For example, a lumen of approximately 0.017 inches may be used with a 0.014 inch wire, and a lumen of approximately 0.020 inches may be used with a 0.018 inch wire. Generally the diameter D3 may be smaller than the diameter D2, but in some embodiments may be larger.

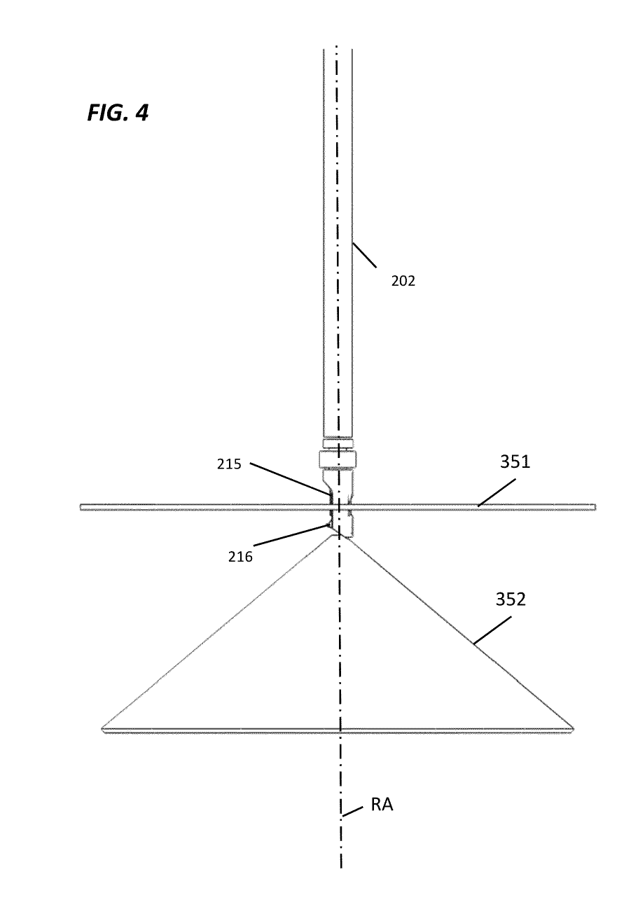

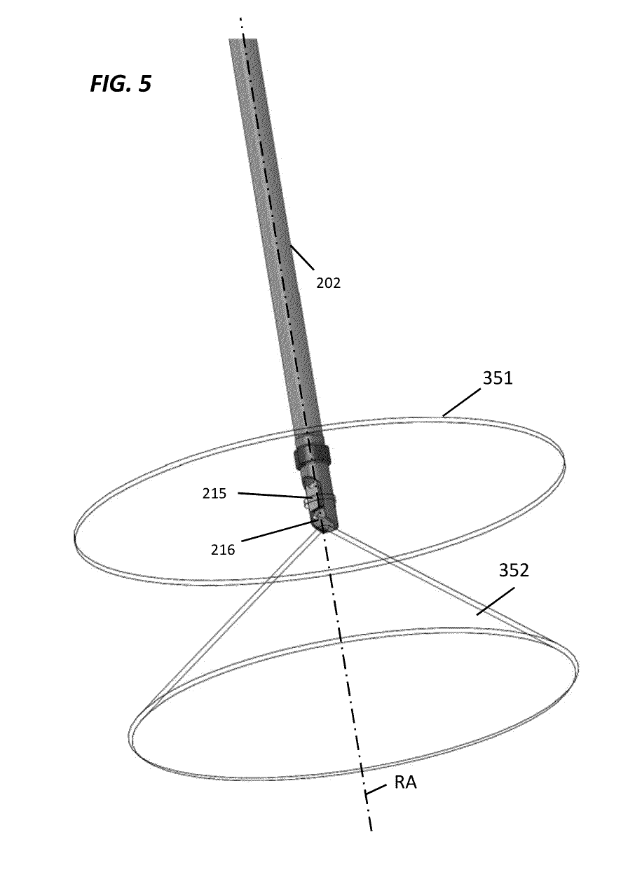

FIGS. 4, 5, and 6 illustrate the imaging core 202. In use, the imaging core 202 is activated to rotate about the rotational axis RA, which causes the transducer subassembly 215 to rotate and generate an imaging cross-sectional ring 351 and which causes the transducer subassembly 216 to rotate and produce an imaging cone 352. The imaging ring 351 is generally perpendicular to the axis of rotation RA. The imaging cone 352 is a 45° imaging cone generated by the rotation of transducer subassembly 216 angled at a mounting angle of 45°. In an alternative embodiment, a transducer subassembly 216 may be mounted at mounting angle of 30° to produce a 30° imaging cone 354. In other alternatives, the transducer subassembly 216 may be mounted at other angles, for example 55°, 35°, 25°, or 15° to generate imaging cones of corresponding sizes. In still other alternatives, the mounting angle may be variable and may be adjusted by an operator prior to or during an imaging procedure.

FIG. 6 further illustrates the imaging core 202 assembled within the sheath 300, with a guidewire 350 extending from the lumen 308 of the sheath. The forward-scanning transducer subassembly 216 transmits and receives through the open distal end 302 of the sheath 300. The IVUS catheter 200 is also able to transmit and receive ultrasound signals with the side-scanning transducer subassembly 215 that transmits and receives perpendicular to the axis of rotation, through the sheath 300.

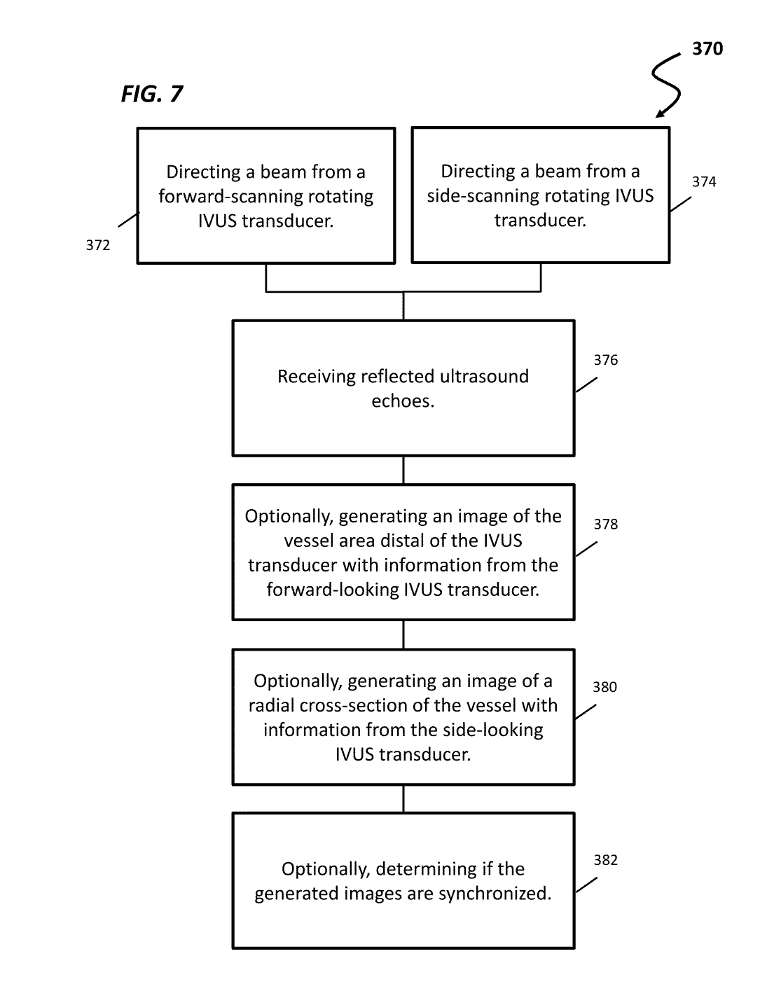

FIG. 7 is a flowchart 370 describing a method of using the catheter system 200. Prior to the implementation of this method, the imaging core 202 is inserted into the sheath 300, and the sheath is guided along a patient's luminated tissue, such as an artery or blood vessel. The sheath 300 is guided, along the guide wire 350, until the tissue to be imaged is positioned distally of the distal end of the sheath. At step 372, an ultrasonic beam is emitted from the transducer subassembly 216 and directed through the distal opening of the sheath 300. The transducer subassembly 216 is rotated about the rotational axis of the imaging core 202 at a rotational speed of approximately 1800 RPM. Slower or faster speeds may also provide effective imaging. The ultrasonic beam encounters the tissue, including any blockages or occlusions, located distally of the sheath. Before, after, or contemporaneously with step 372, an ultrasonic beam is emitted from the transducer subassembly 215 generally perpendicular to the axis of rotation of imaging core 202 and through the sheath 300. The transducer subassembly 215 is rotated about the rotational axis of the imaging core together with rotation of the transducer subassembly 216. The ultrasonic beam from the transducer subassembly 215 encounters tissue, including any blockages or occlusions, located laterally of the sheath. At step 376, reflected ultrasonic echoes are received by the transducer subassemblies 215, 216.

Optionally, at step 378, an image of the tissue located distally of the sheath is generated on a display based on information from the forward-scanning transducer subassembly 216. Because the guide wire 350 may be visible in the generated image, a user may rotate the sheath 300 relative to the interface module. In doing so, the location of the wire 350 in the generated image may be rotated either away from an area of interest to reduce obstruction of view or into an area of interest to serve as a referencing landmark. Optionally, at step 380, an image of a radial cross-section of the vessel is generated on a display based upon information from the side-scanning transducer assembly 215.

Optionally, at step 382, the images from the forward-scanning and side-scanning transducers are displayed on a common display. The synchronization of the two images may provide the clinician with information regarding the position and orientation of the distal end of catheter 200 within the patient vessel.

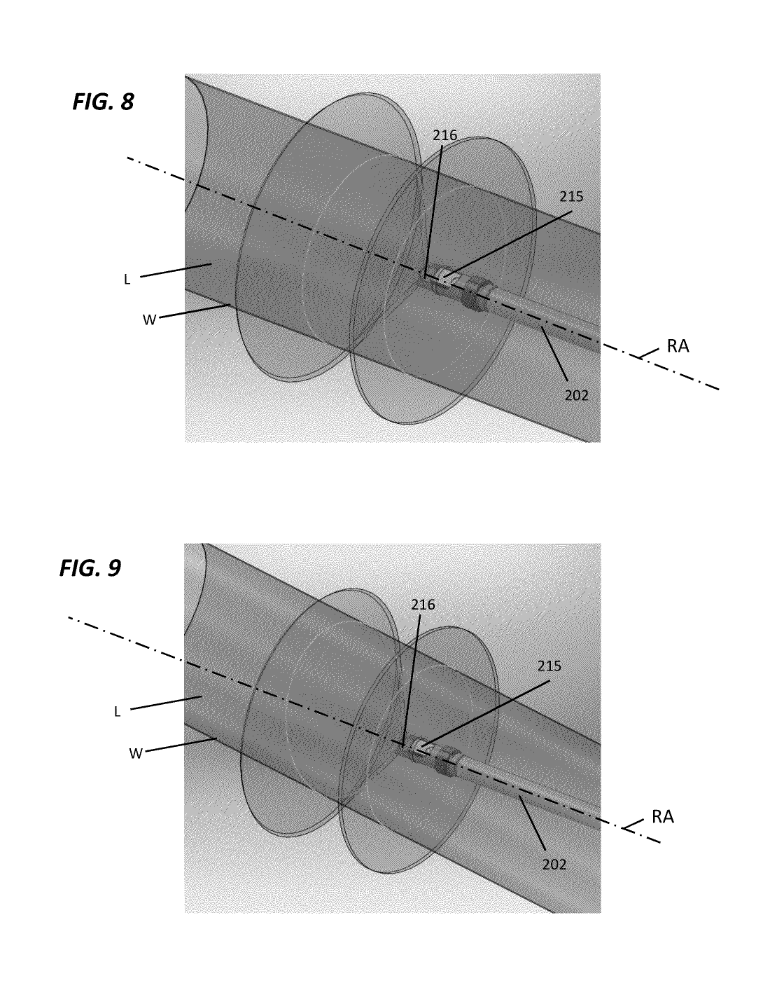

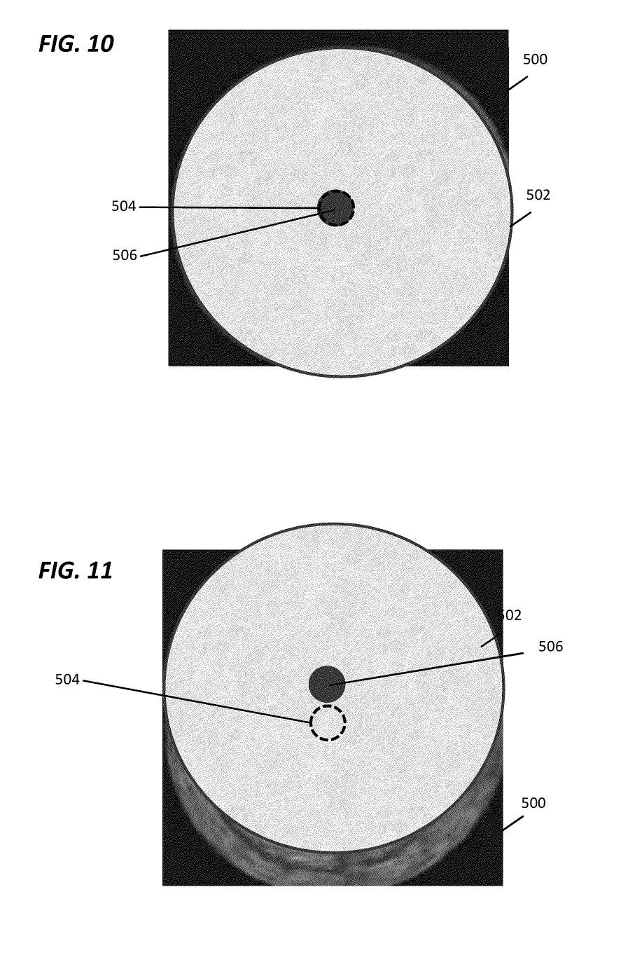

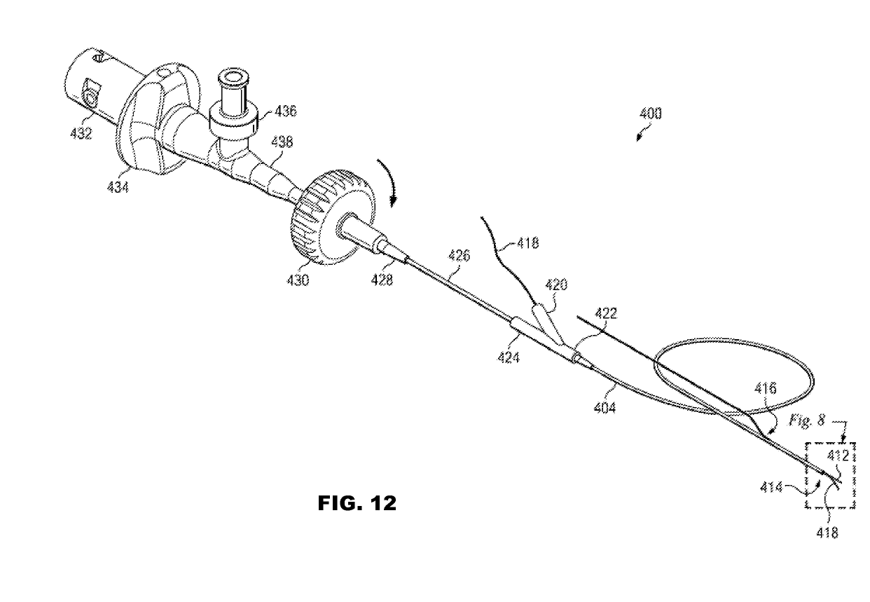

For example, FIG. 8 illustrates the imaging core 202 positioned within a patient lumen L. The imaging core 202 is centrally located within and extending generally parallel to a lumen wall W. With the imaging core 202 positioned as shown in FIG. 8, the images shown in FIG. 10 may be displayed. As shown in FIG. 10, an image 500 from the forward-scanning transducer subassembly 216 and an image 502 from the side-scanning transducer subassembly 215 are displayed on a common display screen. Each of the images 500, 502 may be registered with the axis of rotation at the longitudinal location of the transducer subassembly. The image 500 has a center mark 504 indicating the center of rotation for the forward-scanning transducer 216. The image 502 has a center mark 506 indicating the center of rotation for the side-scanning transducer 215. As shown in FIG. 10, when the images 500, 502 are synchronized, the center marks 504, 506 generally overlap. The synchronized images 500, 502 may indicate to a clinician that the distal tip of the imaging core 202 is positioned within and generally parallel to the wall W of the patient lumen L.

If, as shown in FIG. 9, the imaging core 202 extends within the lumen L at an oblique angle (e.g. 10° in this embodiment) with respect to the lumen wall W, the images 500, 502 (FIG. 11) become separated, with the center marks 504, 506 drifting apart, the images are considered desynchronized. Desynchronized images may indicate to a clinician that the distal tip of the imaging core 202 has become angled within the patient vessel. Desynchronization may also indicate to the clinician that the distal tip of the imaging core 202 is approaching a bend in the lumen L.

Some methods for treating vascular occlusions involve crossing the occlusion with a guide wire prior to opening the occluded vasculature. These methods are true lumen (i.e., natural vascular lumen) crossing techniques and involve passing the guidewire through the occlusion. When crossing the occlusion through the true lumen, the clinician attempts to keep the catheter centered within the vessel lumen. The forward-scanning transducer assembly allows the clinician to visualize the vessel, plaque morphology, and other vessel lumens near the true lumen. The use of dual transducer assemblies provides the clinician with additional information to help the clinician keep the catheter within the true lumen. The dual image display with synchronized or desynchronized images indicates the position and orientation of the distal tip of the catheter with respect to the vessel lumen. When the images become desynchronized, indicating that the distal catheter tip has become angled within the patient vessel, the clinician may initiate deflection changes to the catheter to steer the catheter back to a centered pose. As the catheter is recentered, dual images become re-synchronized.

Sub-intimal crossing is another technique for guide wire placement that involves advancing the guidewire through a sub-intimal passage, tangentially past the occlusion. The guide wire re-enters the true lumen after passing the occlusion. With a single transducer subassembly, re-entry into the true lumen may be challenging with the catheter advancing well beyond the occlusion before re-entry. Using a dual scanning catheter system may facilitate true lumen re-entry by locating and selecting a sub-intimal passage that returns the catheter to the true lumen near the distal side of the occlusion. The dual scanning catheter thus reduces the likelihood of uncontrolled advancement of the catheter, allowing the catheter to re-enter the true lumen immediately after the occlusion has been crossed.

The use of a dual scanning catheter may also provide information to the clinician about the patient's blood flow. With the ability to scan the blood flow at 45° with the forward-scanning transducer subassembly and at 90° with the side-scanning transducer subassembly, the velocity of the blood flow may be calculated. The 45° image provides a Doppler effect for the blood flow. By adding the images together, subtracting the images from one another, or other methods of comparison, the blood flow can be computed. The computed information about the blood flow velocity allows images of relative blood flow to be mapped and imaged. The blood flow velocity information may also allow blood speckle to be removed from the displayed images.

The use of dual scanning also may allow the clinician to determine where the guide wire is located. For example, as the clinician advances the guide wire, with the catheter held relatively stationary, the guide wire is visualized twice, first in the side-scanning image and second in the forward-scanning image. From the images, the clinician may determine the location of the guide wire.

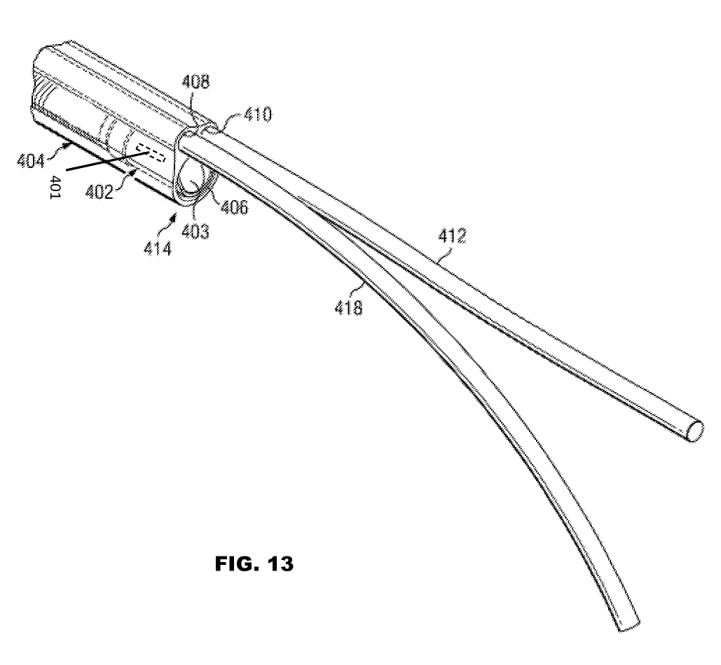

FIGS. 12 and 13 illustrate an IVUS catheter system 400 according to another embodiment of the present disclosure, with FIG. 13 illustrating in greater detail the distal end portion of the system. The system 400 includes an imaging core 402 with a forward-scanning transducer subassembly 403 and a side-scanning transducer subassembly 401. The imaging core 402, the transducer subassembly 401, and the transducer subassembly 403 are the same or substantially similar to the forward-looking imaging core 202, the transducer subassembly 215, and the transducer subassembly 216, respectively as previously described. The system 400 also includes a multi-lumen sheath 404 which is similar to sheath 300, but includes three lumens 406, 408, 410 rather than two. The imaging core 402 extends through and is rotatable within the lumen 406, as previously described for imaging core 202. Lumens 408, 410 are arranged generally adjacent to each other on a common side of the lumen 406. A guide wire 412 extends through lumen 410. The lumen 410 extends between a distal end 414 of the sheath 404 and a medial opening 416 through which the guide wire 412 extends. This type of catheter structure is a “rapid exchange” (RX) catheter. In alternative embodiments, an “over-the-wire” (OTW) catheter structure may be used.

OTW catheters have a lumen that extends inside the entire length of the catheter into which a guide wire can be inserted. With RX catheters, the guide wire only enters into the catheter body near its distal end, instead of entering at the proximal-most end, and extends inside the catheter body to the distal most end of the catheter where it exits.

There are advantages and disadvantages to both designs. The OTW catheters allow easy exchange of guide wires should additional catheter support, from a stiffer guide wire, or a change in the shape or stiffness of the guide wire tip be necessary. The RX catheters allow the operator to more rapidly change from one catheter to another while leaving the guide wire in place, thereby preserving the placement of the guide wire distal tip, which may have been difficult to achieve. Although “standard” length (typically approximately 190 cm) guide wires usually have a proximal extension capability built in (extending the overall length to approximately 300 cm), the use of these accessories is cumbersome and can require two sterile operators.

Typically, about 190 cm guide wires are required to span a vessel from the most distal anatomy that the interventionalist or operator wishes to treat to the point where the guide catheter, enters the patient's body. The entry point may be located, for example, at the femoral artery in a patient's groin, or on occasion, the radial artery in a patient's arm. If the catheter being loaded over the guide wire is an OTW catheter, the guide wire must be long enough so that the entire length of the OTW catheter can be slid over the proximal end of the guide wire and yet there remain some length of the guide wire exposed where it enters the patient's body. That is, the guide wire for an OTW catheter must be approximately twice as long as one that is to be used only with RX catheters, because it must simultaneously accommodate both the length inside the patient's body and the length of the OTW catheter. Further, the “threading” of the OTW catheter over the distal or proximal end of the guide wire may be time consuming, and the added length of the guide wire can be cumbersome to handle while maintaining sterility.

Since the RX design catheters typically have the guide wire running inside them for only the most distal approximately 1 cm to approximately 30 cm, the guide wire employed need only have a little more than the required approximately 1 cm to 30 cm after it exits the patient's body. While the loading of an approximately 140 cm OTW catheter over an approximately 280 cm to 300 cm guide wire is time consuming and tedious, loading the distal approximately 10 cm of an RX catheter over a shorter guide wire is easily done.

However, OTW catheters may track the path of the guide wire more reliably than RX catheters. That is, the guide wire, acting as a rail, prevents buckling of the catheter shaft when it is pushed forward from its proximal end over the guide wire. RX catheters can, however, given a sufficiently wide target site, such as a sufficiently wide artery, and a sufficiently tortuous guide wire path, buckle as they are advanced along the guide wire by pushing on the proximal end of the catheter. In addition, when an RX catheter is withdrawn from a patient, the RX portion can pull on the guide wire and cause the guide wire to buckle near the point that it exits the proximal end of the RX channel.

Referring again to FIGS. 12 and 13 the lumen 408 may be sized for receipt and passage of an elongated flexible shaft or wire 418 carrying an instrument for conducting a therapeutic procedure. For example, the shaft 418 may carry an instrument for reducing artery or vessel blockages, delivering a stent, conducting a biopsy, performing an ablation, delivering an aneurysm graft, conducting an embolization procedure, or draining fluid. The shaft 418 is inserted into the lumen 408 of the sheath 404 via a port 420 of an adaptor 422. The adaptor 422 further includes a port 424 connected to a single-lumen catheter 426. The sheath 404 is coupled via an adaptor 428 to a control knob 430. Rotating the control knob 430 rotates the sheath 404 relative to the interface module and the imaging core 402. Rotation of the sheath 404 allows a user to control the physical location of the guide wire or procedural wire and thus control the position of the image of the guide wire or procedural wire in the resulting IVUS image. In this way, the image of the wires 412, 418 may be moved out of the way of an area requiring analysis or moved into an area of interest to serve as a landmark.

The imaging core 402 is terminated at a proximal end by a rotational interface 432 providing electrical and mechanical coupling to an interface module (See FIG. 1). The IVUS system 400 further includes a hub 434 that supports the rotational interface 432 and provides a bearing surface and a fluid seal between the rotating and non-rotating elements of the catheter assembly. The hub 434 includes a luer lock flush port 436 through which saline may be injected. Saline may provide a biocompatible lubricant for the rotating imaging core 402. The hub 434 is coupled by a tapered adaptor 438 to the control knob 430.

In alternative embodiments, rotating imaging cores may be coupled to more than two transducer subassemblies to generate images from more than two vantage points within the patient vessel.

One or more elements in embodiments of the invention may be implemented in software to execute on a processor of a computer system such as control system 106. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device, The code segments may be downloaded via computer networks such as the Internet, Intranet, etc.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system for imaging a vessel of a patient, the system comprising:
an elongated sheath with proximal and distal ends, the elongated sheath including a flexible body with a first lumen in communication with a distal opening at the distal end; and
an imaging core disposed within the first lumen and rotatable about an axis of rotation, the imaging core including
a first transducer subassembly adapted to transmit a first beam, distally of the flexible body, through the distal opening and
a second transducer subassembly adapted to transmit a second beam through the flexible body in a transverse direction to the axis of rotation.

2. The system of claim 1 wherein the first transducer subassembly includes a planar face extending at an oblique angle with respect to the axis of rotation.

3. The system of claim 2 wherein the oblique angle is approximately 45 degrees.

4. The system of claim 1 wherein the second transducer subassembly includes a planar face extending approximately parallel to the axis of rotation.

5. The system of claim 1 wherein the imaging core includes a shaft supporting both the first and second transducer for rotation about the axis of rotation.

6. The system of claim 1 wherein the flexible body includes a second lumen sized to receive a guide wire.

7. The system of claim 6 wherein the second lumen has a proximal opening between the proximal and distal ends of the elongated sheath and a distal opening at the distal end of the sheath.

8. The system of claim 1 wherein the flexible body includes a third lumen.

9. The system of claim 8 further comprising a steering wire sized for passage within the third lumen.

10. The system of claim 8 further comprising a procedural instrument sized for passage within the third lumen.

11. The system of claim 1 further including a control knob for rotating the elongated sheath relative to the imaging core.

12. The system of claim 1 wherein the first beam is an ultrasound beam.

13. The system of claim 1 wherein the second beam is an ultrasound beam.

14. The system of claim 1 wherein the transducer subassembly is rotatable within the first lumen as bodily fluids from the vessel of the patient are received in the first lumen and engage with the transducer subassembly.

15. A method of imaging a vessel of a patient, the method comprising:
providing an elongated sheath having a proximal end and a distal end and including a flexible body with a first lumen in communication with a distal opening at the distal end;
rotating a first transducer subassembly about an axis of rotation within the first lumen while directing a first beam from the distal opening;
rotating a second transducer subassembly about the axis of rotation within the first lumen while directing a second beam approximately transversely to the axis of rotation; and
receiving reflected first and second beams.

16. The method of claim 15 further comprising generating a first image of an area of the vessel distal of the distal end of the elongated sheath from the reflected first beam and generating a second image of a radial cross-section of the vessel from the reflected second beam.

17. The method of claim 16 further comprising displaying synchronized first and second images when the distal end of the elongated sheath is approximately parallel with a vessel wall of the vessel of the patient.

18. The method of claim 16 further comprising displaying desynchronized first and second images when the distal end of the elongated sheath is positioned at an oblique angle to the vessel wall of the vessel of the patient.

19. The method of claim 15 further comprising measuring the velocity of blood flow with information from the reflected first and second beams.

20. The method of claim 19 further comprising providing a blood flow map image.

21. The method of claim 15 wherein the first transducer subassembly includes a planar face extending at an oblique angle with respect to an axis of rotation of the transducer subassembly.

22. The method of claim 21 wherein the oblique angle is 45 degrees.

23. The method of claim 15 wherein the second transducer subassembly includes a planar face extending approximately parallel to the axis of rotation.

24. The method of claim 15 wherein a common shaft supports the first and second transducer assemblies for rotation about the axis of rotation.

25. The method of claim 15 wherein the flexible body includes a second lumen, the method further comprising providing a guide wire disposed within the second lumen.

26. The method of claim 15 wherein the flexible body includes a third lumen.

27. The method of claim 26 further comprising receiving a steering wire in the third lumen.

28. The method of claim 26 further comprising receiving a shaft of a procedural instrument in the third lumen.

29. The method of claim 15 wherein the first and second beams are ultrasound beams.
